(19) United States
(12) Patent Application Publication
(10) Pub. No.: US 2025/0107712 A1
(43) Pub. Date: Apr. 3, 2025

(54) Acoustic-based imaging device synchronization

(71) Applicant: Ceretype Neuromedicine, Inc., Cambridge, MA (US)

(72) Inventors: Prantik Kundu, Flushing, NY (US); Emily Stern, Chestnut Hill, MA (US)

(21) Appl. No.: 18/847,736

(22) PCT Filed: Mar. 21, 2023
(86) PCT No.: PCT/US2023/015744

§ 371 Date: Sep. 17, 2024

Related U.S. Application Data
(60) Provisional application No. 63/322,173, filed on Mar. 21, 2022.

(51) Int. Cl. A61B 5/00; A61B 5/0533; A61B 5/055; A61B 5/246; A61B 5/377; G01R 33/28; G01R 33/48

(52) U.S. Cl. CPC A61B 5/0042; A61B 5/0533; A61B 5/055; A61B 5/7289; G01R 33/28; G01R 33/4806; A61B 5/246; A61B 5/377

(57) ABSTRACT

Medical imaging systems and methods. The systems include a sound detection device configured to detect an acoustic feature associated with the operation of an imaging device and an interface configured to present a stimulus to a patient. The system also includes at least one processor executing instructions stored on memory to record feedback from the patient in response to the presented stimulus, receive imagery of the patient from the imaging device, identify feedback provided by the patient coinciding with the detection of the acoustic feature, identify imagery of the patient coinciding with the detection of the acoustic feature, and supply the identified feedback and identified imagery to an operator.

### CROSS REFERENCE TO RELATED APPLICATIONS

The present application claims the benefit of and priority to co-pending U.S. provisional application No. 63/322,173, filed on Mar. 21, 2022, the content of which is hereby incorporated by reference as if set forth in its entirety herein.

### TECHNICAL FIELD

Embodiments described herein generally relate to systems and methods for gathering imagery of a patient and, more specifically but not exclusively, to systems and methods for operating imagery gathering devices.

### BACKGROUND

Treating various medical ailments may involve gathering brain imaging data to monitor aspects of a patient's behavior, consciousness, mental health, brain health, or the like. Modalities, devices, or systems (for simplicity, “imaging devices”) for gathering brain imaging data include, but are not necessarily limited to, magnetic resonance imaging (MRI) devices, positron emission tomography (PET) devices, near infrared spectroscopy (NIRS) devices, electro-encephalography (EEG) devices, and magneto-encephalography (MEG) devices.

During patient monitoring, it may be beneficial to modulate the patient's brain state over time, such as by having the patient alternate between a task stage and a rest stage. For example, an examination or monitoring procedure may be designed to alternate a patient's brain state between a task state and a rest state over a time period spanning seconds or hours. This helps gather meaningful imagery such as of a specific brain region that is associated with the task or rest stage.

Characterizing neural substrates during various behaviors is benefitted by synchronizing the imaging modality and the neuromodulation system (e.g., a system designed to solicit patient feedback). This synchronization is typically achieved by communicating signals between the imaging device and the neuromodulation modality. These signals may include signals sent over conductive cabling, optical signals sent over fiber-optic cabling, or radio-frequency signals sent between transmitters and receivers that are interconnected with the imaging device and neuromodulation system mentioned above.

Synchronizing these systems typically requires configuring software or physically manipulating system components. These components, however, may be sensitive or susceptible to damage if improperly handled. Proper handling or configuration of these components is therefore time consuming, resource intensive, and requires training of personnel to make the required configuration changes.

A need exists, therefore, for systems and methods that overcome the disadvantages associated with existing techniques for synchronizing these types of systems.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a medical imaging system. The system includes a sound detection device configured to detect an acoustic feature associated with operation of an imaging device; an interface configured to present a stimulus to a patient; and at least one processor executing instructions stored on memory to record feedback from the patient in response to the presented stimulus, receive imagery of the patient from the imaging device, identify feedback provided by the patient coinciding with the detection of the acoustic feature, identify imagery of the patient coinciding with the detection of the acoustic feature, and supply the identified feedback and identified imagery to an operator.

In some embodiments, the acoustic feature associated with the operation of the imaging device relates to electromagnetic modulation of the imaging device.

In some embodiments, the system includes a response recorder device accessible by the patient during operation of the imagery device, the response recorder device configured to record feedback provided by the patient. In some embodiments, the response recorder device further includes the sound detection device. In some embodiments, the response recorder device is configured to provide the feedback from the patient to the at least one processor after the imagery is gathered by the imaging device. In some embodiments, the imaging device is a magnetic resonance imaging device, and the response recorder device includes a sensor to detect a change in a gradient magnetic field based on an acoustic feature indicating gradient switching. In some embodiments, the at least one processor is further configured to identify coinciding feedback using the detected change in the gradient magnetic field.

In some embodiments, the recorded feedback from the patient is a galvanic skin response, an electrophysiological measurement of the patient, or a patient activation of an input component.

In some embodiments, the received imagery of the patient includes imagery of the patient's brain, and the imaging device includes at least one or more of a magnetic resonance device, a positron emission tomography device, a near infrared spectroscopy device, an electro-encephalography imaging device, or a magneto-encephalography imaging device.

According to another aspect, embodiments relate to a response recorder device. The response recorder device includes a sound detection device configured to detect an acoustic feature associated with operation of an imaging device, an input module to record feedback from a patient in response to the patient being presented with a stimulus, a storage module configured to store data associated with the acoustic feature and the recorded feedback, and an interface in operable communication with an external system and configured to communicate the stored data associated with the acoustic feature and the recorded feedback to the external system.

In some embodiments, the feedback coincides with the detection of the acoustic feature.

In some embodiments, the system further includes at least one processor executing instructions stored on memory to process the data associated with the acoustic feature and the recorded feedback prior to the interface communicating the acoustic feature and the recorded feedback to the external system.

In some embodiments, the recorded feedback from the patient is a galvanic skin response, an electrophysiological measurement of the patient, or a patient activation of an input component.

According to another aspect, embodiments relate to a method of operating a medical imaging system. The method includes presenting a stimulus to a patient using an interface, recording a patient feedback to the stimulus, gathering imagery of the patient using an imaging, gathering an acoustic feature associated with operation of the imaging device, and identifying feedback and imagery coinciding with the gathered acoustic feature.

In some embodiments, the acoustic feature associated with the operation of the imaging device relates to electromagnetic modulation of the imaging device.

In some embodiments, the recorded feedback from the patient is a galvanic skin response, an electrophysiological measurement of the patient, or a patient activation of an input component.

In some embodiments, the gathered imagery of the patient includes imagery of the patient's brain, and the imaging device includes at least one of a magnetic resonance device, a positron emission tomography device, a near infrared spectroscopy device, an electro-encephalography, or a magneto-encephalography imaging device.

In some embodiments, the patient's response is recorded by a response recorder device operational in proximity to a magnetic field produced by a magnetic resonance imaging device.

In some embodiments, the method further includes communicating the identified feedback and imagery coinciding with the gathered acoustic feature.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a front view of a response recorder device in accordance with one embodiment;

FIG. 2 illustrates a circuit board of the response recorder device of FIG. 1 in accordance with one embodiment;

FIG. 3 illustrates a medical imaging system in accordance with one embodiment;

FIG. 4 depicts a switching gradient waveform associated with an MRI device in accordance with one embodiment; and

FIG. 5 depicts a flowchart of a method for operating an imagery system in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

As discussed previously, the administration of medical imaging procedures benefits from synchrony between the imaging device and stimulus presentation module. Existing techniques for achieving this synchrony generally rely on configuring complex software or rely on manually manipulating components associated with at least the imaging device. This exposes an operator to injury and risks damaging equipment.

The embodiments herein provide novel techniques for synchronizing an imaging device and a stimulus presentation device. The embodiments herein rely on one or more acoustic features (i.e., sound) produced by an imaging device as part of its operation during a monitoring procedure.

During the monitoring procedure, a patient may have access to a response recorder device to which they can provide feedback in response to a stimulus. For example, the patient may hold the response recorder device and activate an input portion (e.g., one or more buttons) as feedback in response to a presented stimulus. The response recorder device may also include a sound detection device to detect acoustic feature(s) generated by the imaging device.

The response recorder device may communicate data regarding the detected acoustic feature(s) and the patient feedback to one or more locations for analysis. An analysis may involve, for example, identifying the operation state of the imaging device at a time that coincides with an instance of feedback from the patient. Additionally or alternatively, the gathered data may be communicated to an operator for review.

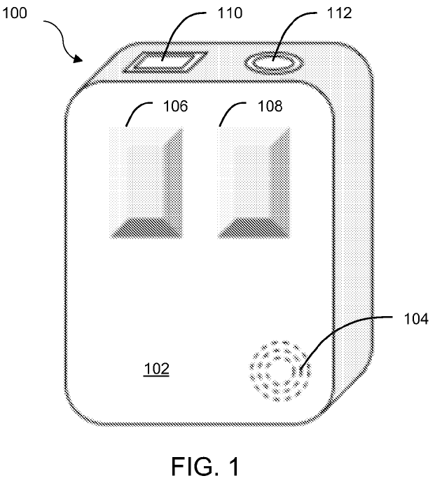

FIG. 1 illustrates a front view of a response recorder device 100 in accordance with one embodiment. The response recorder device 100 may be handled or otherwise accessible by a patient during an examination or monitoring (for simplicity, “monitoring”) procedure. For example, a patient may hold the response recorder device 100 while an imaging device is gathering imagery of the patient's brain. As another example, the response recorder device 100 may be mounted or secured at a location accessible by the patient during the monitoring procedure.

As the response recorder device 100 is in proximity to an imaging device, the response recorder device 100 should comprise components that are not affected by the operation of the imaging device. In other words, the response recorder device 100 should have bio-compatibility with the imaging device and the biophysical principles under which the imaging device is intended to operate.

For example, if the imaging modality is an MRI device, the response recorder device 100 should be safe and operational in the presence of a strong magnetic field. This may require the response recorder device 100 to be built without ferromagnetic components due to the induction force that would occur in such components in the presence of a magnetic field.

However, the response recorder device 100 should also avoid non-ferromagnetic metal components that would be heated in the presence of a changing magnetic field. If these components are heated, they may burn the patient or other personnel if contacted. Additionally, these components may become overheated and not function as desired.

The response recorder device 100 may include a housing 102, a sound detection cover portion 104, one or more input portions (e.g., buttons) 106 and 108, a first port 110, and a second port 112. The housing 102 may be formed from any suitable material and sized such that a patient can hold the housing 102 in their hand(s). Additionally, the housing 102 may be formed from a material that is not adversely affected by the operation of an imaging device used in a monitoring procedure.

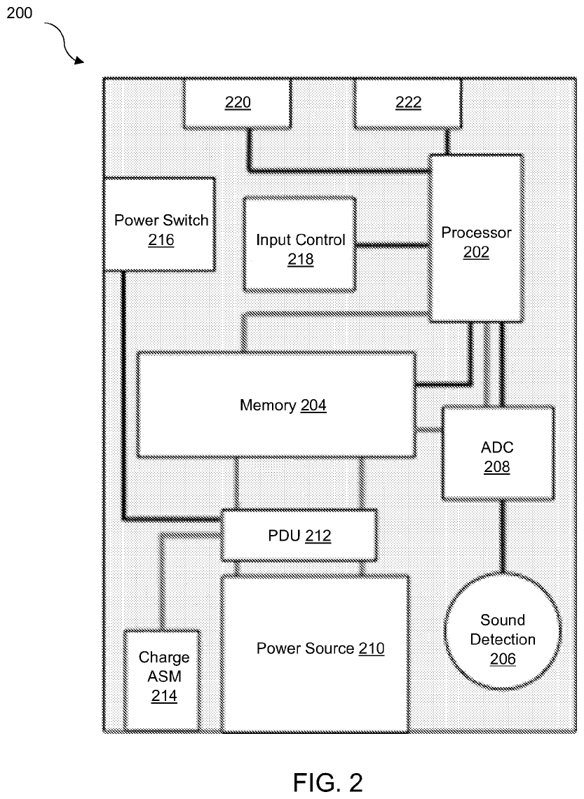

The sound detection cover portion 104 may include one or more apertures to allow the response recorder device 100 to detect an acoustic feature associated with an imaging device. For example, the sound detection cover portion 104 may be positioned over a sound detection device such as a microphone (discussed below in conjunction with FIG. 2). During operation, the microphone may detect sound associated with the operation of the imaging device.

The first input portion 106, second input portion 108, or both, may allow a patient to provide feedback as part of a neuromodulation exercise. For example, the patient may be instructed to activate the second input portion 108 when presented with a certain stimulus. The first input portion 106, second input portion 108, or both, may be configured in a variety of ways. For example, and without limitation, in the input portion(s) may be configured as buttons (as illustrated in FIG. 1), switches, touchscreen portions, or the like. Although two input portions are illustrated in FIG. 1, the response recorder device may include more than two input portions or less than two input portions.

In some embodiments, the response recorder device 100 need not rely on an intentional act by the patient. For example, in some embodiments, the input portion 106 may comprise a sensor to detect an electrophysiological measurement of the patient. As another example, the input portion 106 or 108 may be configured to detect a galvanic skin response, which may constitute an input.

The first port 110 may enable connectivity with an external device or location such as a personal computer (PC). For example, the first outlet port may receive a cable or wire that connects the response recorder device 100 to the PC (not shown in FIG. 1). This connection may provide data regarding, for example, sound detected by the response recorder device and activation of the input portion(s) 106 or 108. This data may be communicated to the external PC in substantially real time, in which case the response recorder device 100 and the external PC are physically connected during the monitoring procedure.

Alternatively, the response recorder device 100 may not be physically connected to an external PC during the monitoring procedure. For example, the response recorder device 100 may gather data related to activation of the input portions 106 or 108, and data regarding sound generated by the imaging device during the monitoring procedure. The response recorder device 100 may store this data locally. After the monitoring procedure is completed, the response recorder device 100 may be connected to the external PC to transmit the gathered data.

The second port 112 may enable operable connectivity with a stimulus system (not shown in FIG. 1). For example, the response recorder device 100 may physically connect to a stimulus system that is in the same room as an imaging device or in a different room (e.g., on the other side of a shield wall or Faraday cage). The stimulus system may be configured to, for example, present a visual stimulus on a screen to the patient, and the patient may be tasked with responding to the stimulus by activating the input portion 106.

Visual stimuli may involve an interactive task in which stimuli appear and disappear with figures, text, sound, or some combination thereof, as cued according to a schedule and cleared (or not) according to one or more input presses. These stimuli may be presented in an arbitrary or non-arbitrary order. For example, a visual stimulus may use or include text and/or graphics to instruct the subject being imaged to press an input button repeatedly according to an indicated pace. The device may record and synchronize the presses with the timing of the acoustic signals and/or transfer the data in near-real time to the stimulus module to, for example, either modify the stimulus or not.

Alternatively, a subject being imaged may be presented with a picture of a person's face or other object, and asked to identify whether the stimulus is a person or not. The subject may press a certain button to indicate their answer. b Other stimuli may include auditory; tactile; or even chemical, as introduced into the patient through an intravenous administration or orally; or some combination thereof.

FIG. 2 illustrates a circuit board 200 associated with the response recorder device 100 of FIG. 1 in accordance with one embodiment. The board 200 may be contained within the housing 102 of FIG. 1, for example.

The board 200 may include, inter alia, at least one processor 202 executing instructions stored on memory 204, a sound detection device 206, an analog-to-digital converter (ADC) 208, and a power source 210. The processor 202 be any hardware device capable of executing instructions stored on memory 204 to process data received from or otherwise associated with the other components of the board 200. The processor 202 may include a microprocessor, a field programmable gate array (FPGA), an application-specific integrated circuit (ASIC), or other similar devices. In some embodiments, such as those relying on one or more ASICs, the functionality described as being provided in part via software may instead be configured into the design of the ASICs and, as such, the associated software may be omitted.

The memory 204 may be L1, L2, L3 cache, or RAM memory configurations. The memory 204 may include non-volatile memory such as flash memory, EPROM, EEPROM, ROM, and PROM, or volatile memory such as static or dynamic RAM, as discussed above. The exact configuration/type of memory 204 may vary as long as instructions for recording and/or processing data regarding gathered acoustic feature(s) and patient feedback can be executed. Any writeable form of memory may also be capable of recording the recorded (“raw”) auditory signals, converted digital indicators, and input signals (i.e., buttons) of a sufficiently long duration as to correspond to one or more monitoring procedures.

The sound detection device 206 may be positioned in proximity to or “below” the sound detection cover portion 104 of FIG. 1 when the response recorder device 100 is in an assembled state. The sound detection device 206 may be configured as a microphone, for example, and may be configured to detect one or more acoustic features related to the operation of an imaging device. The ADC 208 may convert an analog signal from the sound detection device 206 to a digital signal for processing by the processor(s) 202.

Although the sound detection device 206 is located within the housing 102, the embodiments described herein may rely on other types of sound detection devices including those at different locations. For example, the embodiments herein may use an external sound detection device such as a corded or wireless microphone that can be operably connected to a response recorder device.

The power source 210 may include a battery sufficient to provide power to the other components of the board 200. In some embodiments, the power source may be a rechargeable battery.

The power source 210 may be in communication with a power distribution unit (PDU) 212 and a charge assembly 214. The charge assembly 214 and the PDU 212 may control and distribute power from the power source 210 to other components of the board 200. For example, the power source 210 and the PDU 214 may transmit power to certain components of the board 200 via a +/−3V rail.

The board 200 may also include a power switch 216 to turn the response recorder device 100 on and off. For example, the response recorder device 100 may be in an off state to preserve battery life when not being used in a monitoring procedure or being used to transfer data to an external system. A patient or medical personnel may turn the response recorder device 100 on before a monitoring procedure.

The board 200 may also include one or more input controls 218 corresponding to the input portions 106 or 108 of FIG. 1. The input control 218 may detect when a patient activates one or more of the input portions 106 and 108, and communicate data regarding the activation to the processor 202. The input control 218 may also provide data such as the time of an activation to the processor 202.

The first interface 220 may correspond to the first port 110 of FIG. 1. For example, the first interface 220 may enable connection with an external device or location such as a personal computer (PC). The second interface 222 may correspond to the second port 112 of FIG. 1. For example, the second interface 220 may enable connection with a neuromodulation system or stimulus module.

The board 200 may also include a sensor to detect other parameters associated with the imaging device. For example, the board 200 may sense the strength of a magnetic field generated by an MRI using a magnetometer. Additionally or alternatively, this type of sensor may be located separate from the response recorder device.

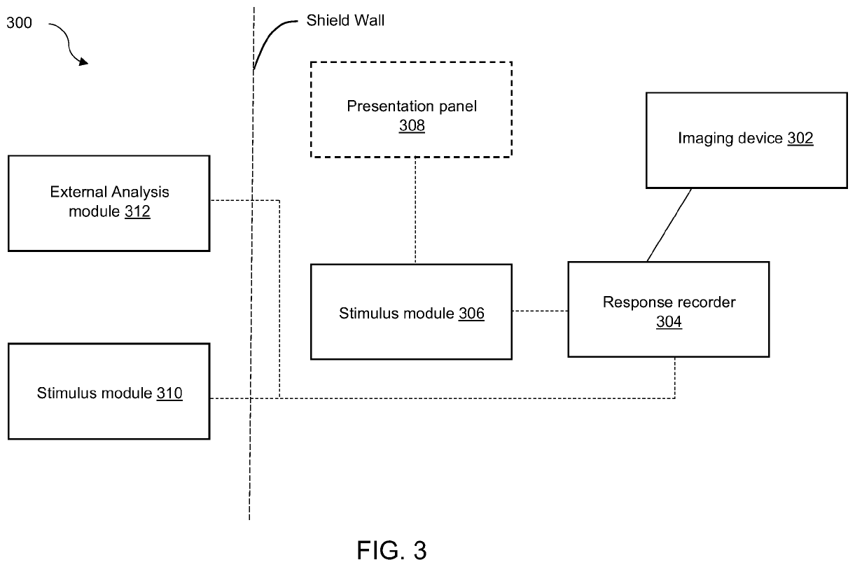

FIG. 3 illustrates a medical imaging system 300 in accordance with one embodiment. The system 300 may include an imaging device 302, a response recorder device 304, a local stimulus module 306 or neuromodulation response module (for simplicity, “first stimulus module 306”) configured to present a stimulus on a presentation panel 308, an exterior stimulus module 310 or a neuromodulation response module (for simplicity “second stimulus module 310”), and an external analysis module 312. The response recorder device 304 may be similar to the response recorder device 100 of FIG. 1 and include the components of the circuit board 200 of FIG. 2.

The system 300 provides a novel technique for synchronizing equipment of a brain monitoring procedure that at least reduces the need to configure or manipulate signaling components of the imaging device 302 or the stimulus module(s) 306, 308. The system 300 relies on one or more acoustic features associated with the operation of the imaging device 302 and the processing of patient feedback provided in response to a stimulus, such as one provided as part of a neuropsychological experiment. For example, the neuropsychological experiment may involve a monitoring procedure to ascertain neural substrates of healthy or pathological behavior displayed by a patient.

Accordingly, the system 300 can synchronize the imaging device 302 with a stimulus module 306, 308 without requiring an explicit configuration or material manipulation of their associated components. As discussed previously, improper handling of such components may be dangerous to operators or may damage the components.

Rather, the embodiments herein use knowledge regarding the operation state to synchronize patient image data with the recording of feedback provided by the patient, the presentation or control of a stimulus, or both. This enables the quick and safe synchronization between devices for neuropsychological experiments involving medical imaging devices and stimulus modules.

In operation of the system 300, the response recorder device 304 of FIG. 3 may detect one or more acoustic features due to the operation of the imaging device 302. For example, a sound detection device such as the sound detection device 206 of FIG. 2 may detect the sound(s) or acoustic features generated by the imaging device 302.

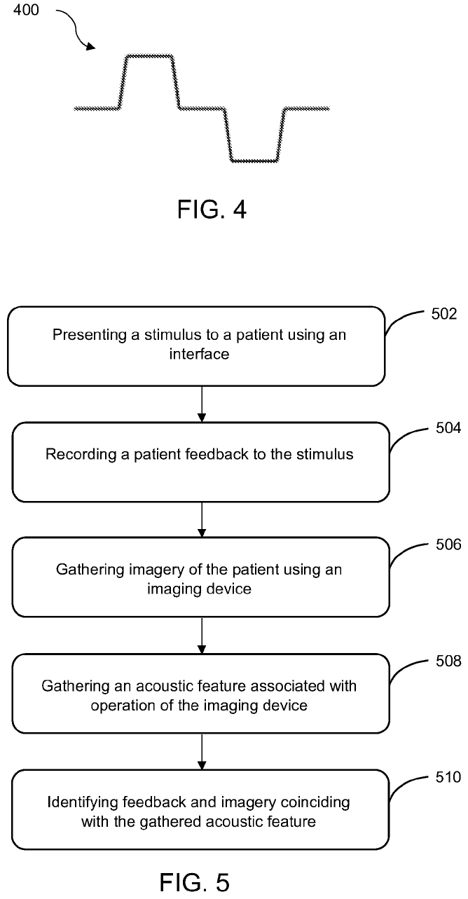

Imaging devices may make repeating patterns of acoustic noise as part of their operation. For example, if the imaging device 302 is an MRI device, the imaging device 302 may produce a high-volume noise in highly repetitive patterns due to the physical mechanism(s) involved in conducting the imaging. For an MRI, these noises arise due to the vibration of physical components resulting from the magneto-electronic switching of an integrated gradient electromagnetic coil. This coil is required to modulate the magnetic field that is the basis for the image gathering procedure. For example, FIG. 4 illustrates a diagram 400 showing a switching gradient waveform associated with an MRI. This waveform would produce a unique acoustic feature detectable by the response recorder device 304.

MRI devices also produce a high-volume noise due to the precise control of repetitive electromagnetic modulations used to tune the time-varying MRI magnetic field. This is needed for sensitive detection and imaging of one or more biophysical processes that manifest in the patient.

Some imaging devices such as electroencephalogram (EEG) scanners or magnetoencephalography (MEG) scanners may not generate a detectable acoustic feature as part of its normal operation. However, in some embodiments the imaging device may be configured to incorporate an acoustic feature such as a ping as part of one or more stages of its operation or otherwise to trigger an event.

During the monitoring procedure, the first stimulus module 306 or second stimulus module 310 present a stimulus to the patient or otherwise perform an action intended to solicit a physiological response from the patient. For example, the stimulus may be intended to at least manipulate the state of the patient's brain. As seen in FIG. 3, the stimulus module 306 may be located proximal to the imaging device and the patient, such as in the same room. In some embodiments, an external stimulus module 310 may be located separate from the imaging device 302 such as in a different room or behind a shield wall or Faraday cage.

The patient may, as part of the monitoring procedure and while being imaged, provide some type of feedback in response to the stimulus. For example, and as discussed previously, the patient may activate an input portion on the response recorder device 304. Additionally or alternatively, the patient feedback may comprise a galvanic skin response or an electrophysiological measurement of the patient.

Data regarding the detected acoustic features may be stored locally in memory of the response recorder device 304. Additionally, data regarding recorded patient feedback may also be stored locally in memory of the response recorder device. During or after the monitoring procedure, the response recorder device 304 may transmit gathered data to an external analysis module 312.

FIG. 5 depicts a flowchart of a method 500 for operating a medical imaging system in accordance with one embodiment. The devices of any one of FIGS. 1-3 or components thereof may be involved with the steps of method 500.

Step 502 involves presenting a stimulus to a patient using an interface. The interface may relate to a stimulus module such as the stimulus modules 306, 310 of FIG. 3. The stimulus may involve a visual stimulus presented on a presentation panel 308 such as a projector screen. In some embodiments, the interface may be configured or designed to present a stimulus intended to solicit an electrophysiological measurement of the patient.

Step 504 involves recording a patient feedback to the stimulus. For example, the patient may be holding or otherwise have access to a response recorder device as discussed in connection with FIGS. 1-3. During a monitoring procedure, the patient may, for example, activate an input portion such as by pressing one or more buttons configured with the response recorder device. This activation may indicate they acknowledge a presented stimulus or have a certain feeling toward a presented stimulus, for example. In some embodiments, the feedback may be an electrophysiological measurement of the patient, a galvanic skin response (e.g., due to sweating), or the like.

Step 506 involves gathering imagery of the patient using an imaging device. The gathered imagery may relate to the patient's brain state, for example. The imaging device may include one or more of a magnetic resonance device, a positron emission tomography device, a near infrared spectroscopy device, an electro-encephalography imaging device, and a magneto-encephalography imaging device.

Step 508 involves gathering an acoustic feature associated with operation of the imaging device. The response recorder device may be configured with a sound detection device such as a microphone as discussed previously.

Imaging devices tend to, during operation, make a series of sounds that can be indicative of the operation state of the imaging device. The sound detection device, which may be configured with or separate from the response recorder device, may gather one or more acoustic features associated with the operation of the imaging device.

Audible tones produced by neuropsychological presentation/modulation equipment using a sound generator (e.g., speaker system) constitute one kind of acoustic feature interpretable by the disclosed embodiments. PET PET—and computed tomography (CT)-based devices may incorporate an automated message for “Breath hold” or other commands into the imaging sequence. These types of commands may also be potential acoustic features.

Step 510 involves identifying feedback and imagery coinciding with the gathered acoustic feature. Data regarding the acoustic feature(s) and the received patient feedback may be communicated to a processing engine such as the external analysis module 310 of FIG. 3.

The identified feedback may be communicated to an operator such as medical personnel conducting the monitoring procedure or reviewing the results of the monitoring procedure. For example, the external analysis module 312 may perform a synchronization procedure to match acoustic features with feedback received from the patient. The external analysis module 312 may output synchronized data to an operator via a user interface, for example.

In some embodiments, the synchronization procedure or other type of analysis may be performed locally by the response recorder device. In some embodiments, the synchronization procedure or other type of analysis may be performed at a location separate from the response recorder device. For example, the response recorder device may communicate the gathered data (e.g., acoustic feature(s), patient feedback, etc.) to a location external to the response recorder device for processing. This data transmittal may be in substantially real time as the data is being gathered during the monitoring procedure. Alternatively, the transmission may occur post hoc after the monitoring procedure has concluded (e.g., upon or after the response recorder device stops recording data).

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. The systems and methods involving hardware and software and/or functional parts therefore may be physically integrated into or housed inside or attached to another device, be it an imaging device, a stimulus or electrophysiological recording device, and patient audio device, etc. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A medical imaging system comprising:
a sound detection device configured to detect an acoustic feature associated with operation of an imaging device;
an interface configured to present a stimulus to a patient; and
at least one processor executing instructions stored on memory to:
record feedback from the patient in response to the presented stimulus,
receive imagery of the patient from the imaging device;
identify feedback provided by the patient coinciding with the detection of the acoustic feature;
identify imagery of the patient coinciding with the detection of the acoustic feature; and
supply the identified feedback and identified imagery to an operator.

2. The system of claim 1 wherein the acoustic feature associated with the operation of the imaging device relates to electromagnetic modulation of the imaging device.

3. The system of claim 1 further comprising a response recorder device accessible by the patient during operation of the imaging device, the response recorder device configured to record feedback provided by the patient.

4. The system of claim 3 wherein the response recorder device further includes the sound detection device.

5. The system of claim 3 wherein the response recorder device is configured to provide the feedback from the patient to the at least one processor after the imagery is gathered by the imaging device.

6. The system of claim 3 wherein the imaging device is a magnetic resonance imaging device, and the response recorder device includes a sensor to detect a change in a gradient magnetic field based on an acoustic feature indicating gradient switching.

7. The system of claim 6 wherein the at least one processor is further configured to identify coinciding feedback and imagery using the detected change in the gradient magnetic field.

8. The system of claim 1 wherein the recorded feedback from the patient is a galvanic skin response, an electrophysiological measurement of the patient, or a patient activation of an input component.

9. The system of claim 1 wherein the received imagery of the patient includes imagery of the patient's brain, and the imaging device includes at least one or more of a magnetic resonance device, a positron emission tomography device, a near infrared spectroscopy device, an electro-encephalography imaging device, or a magneto-encephalography imaging device.

10. A response recorder device comprising:
a sound detection device configured to detect an acoustic feature associated with operation of an imaging device;
an input module to record feedback from a patient in response to the patient being presented with a stimulus;
a storage module configured to store data associated with the acoustic feature and the recorded feedback; and
an interface in operable communication with an external system and configured to communicate the stored data associated with the acoustic feature and the recorded feedback to the external system.

11. The response recorder device of claim 10 wherein the feedback coincides with the detection of the acoustic feature.

12. The response recorder device of claim 10 further comprising at least one processor executing instructions stored on memory to process the data associated with the acoustic feature and the recorded feedback prior to the interface communicating the acoustic feature and the recorded feedback to the external system.

13. The response recorder device of claim 10 wherein the recorded feedback from the patient is a galvanic skin response, an electrophysiological measurement of the patient, or a patient activation of an input component.

14. A method of operating a medical imaging system, the method comprising:
presenting a stimulus to a patient using an interface;
recording a patient feedback to the stimulus;
gathering imagery of the patient using an imaging device;
gathering an acoustic feature associated with operation of the imaging device; and
identifying feedback and imagery coinciding with the gathered acoustic feature.

15. The method of claim 14 wherein the acoustic feature associated with the operation of the imaging device relates to electromagnetic modulation of the imaging device.

16. The method of claim 14 wherein the recorded feedback from the patient is a galvanic skin response, an electrophysiological measurement of the patient, or a patient activation of an input component.

17. The method of claim 14 wherein the gathered imagery of the patient includes imagery of the patient's brain, and the imaging device includes at least one of a magnetic resonance device, a positron emission tomography device, a near infrared spectroscopy device, an electro-encephalography, or a magneto-encephalography imaging device.

18. The method of claim 14 wherein the patient's response is recorded by a response recorder device operational in proximity to a magnetic field produced by a magnetic resonance imaging device.

19. The method of claim 14 further comprising communicating the identified feedback and imagery coinciding with the gathered acoustic feature.
